(19) United States
(12) Patent Application Publication
(10) Pub. No.: US 2020/0392458 A1
(43) Pub. Date: Dec. 17, 2020

(54) MODIFIED NATURAL KILLER CELLS AND NATURAL KILLER CELL LINES TARGETTING TUMOUR CELLS

(71) Applicant: ONK THERAPEUTICS LIMITED, Galway (IE)

(72) Inventors: Michael Eamon Peter O'DWYER, Galway (IE); Subhashis SARKAR, Galway (IE)

(21) Appl. No.: 16/766,652

(22) PCT Filed: Nov. 26, 2018
(86) PCT No.: PCT/EP2018/082556

§ 371 Date: May 22, 2020

(30) Foreign Application Priority Data
Nov. 24, 2017 (EP) 17203649.3

(51) Int. Cl. C12N 5/0783; G01N 33/50; G01N 33/574; A61K 35/17; A61K 38/17; A61K 39/00

(52) U.S. Cl. CPC C12N 5/0646; G01N 33/5047; G01N 33/57492; A61K 35/17; A61K 2039/5156; A61K 39/00117; C12N 2510/00; G01N 2333/4725; A61K 38/1774

(57) ABSTRACT

NK cells and NK cell lines are modified to increase their selectivity for cancer cells by providing an ability to bind tumour associated MUC-1 antigen. Production of such modified NK cells and NK cell lines is via genetic modification to produce NK-CARs that are optionally further modified to have increased cytotoxicity against cancer cells.

### INTRODUCTION

The present invention relates to the modification of natural killer (NK) cells and NK cell lines to produce derivatives thereof with a more cytotoxic phenotype. Furthermore, the present invention relates to methods of producing modified NK cells and NK cell lines, compositions containing the cells and cell lines and uses of said cells, lines and compositions in the treatment of cancer, especially blood cancers.

### BACKGROUND TO THE INVENTION

Typically, immune cells require a target cell to present antigen via major histocompatibility complex (MHC) before triggering an immune response resulting in the death of the target cell. This allows cancer cells not presenting MHC class I to evade the majority of immune responses.

NK cells are able, however, to recognize cancer cells in the absence of MHC class I expression. Hence they perform a critical role in the body's defence against cancer.

On the other hand, in certain circumstances, cancer cells demonstrate an ability to dampen the cytotoxic activity of NK cells, through expression of ligands that bind inhibitory receptors on the NK cell membrane. Resistance to cancer can involve a balance between these and other factors.

Cytotoxicity, in this context, refers to the ability of immune effector cells, e.g. NK cells, to induce cancer cell death, e.g. by releasing cytolytic compounds or by binding receptors on cancer cell membranes and inducing apoptosis of said cancer cells. Cytotoxicity is affected not only by signals that induce release of cytolytic compounds but also by signals that inhibit their release. An increase in cytotoxicity will therefore lead to more efficient killing of cancer cells, with less chance of the cancer cell dampening the cytotoxic activity of the NK, as mentioned above.

Acute myeloid leukemia (AML) is a hematopoietic malignancy involving precursor cells committed to myeloid development, and accounts for a significant proportion of acute leukemias in both adults (90%) and children (15-20%) (Hurwitz, Mounce et al. 1995; Lowenberg, Downing et al. 1999). Despite 80% of patients achieving remission with standard chemotherapy (Hurwitz, Mounce et al. 1995; Ribeiro, Razzouk et al. 2005), survival remains unsatisfactory because of high relapse rates from minimal residual disease (MRD). The five-year survival is age-dependent; 60% in children (Rubnitz 2012), 40% in adults under 65 (Lowenberg, Downing et al. 1999) and 10% in adults over 65 (Ferrara and Schiffer 2013). These outcomes can be improved if patients have a suitable hematopoietic cell donor, but many do not, highlighting the need for an alternative approach to treatment.

NK cells are cytotoxic lymphocytes, with distinct phenotypes and effector functions that differ from e.g. natural killer T (NK-T) cells. For example, while NK-T cells express both CD3 and T cell antigen receptors (TCRs), NK cells do not. NK cells are generally found to express the markers CD16 and CD56, wherein CD16 functions as an Fc receptor and mediates antibody dependent cell-mediated cytotoxicity (ADCC) which is discussed below. KHYG-1 (see below) is a notable exception in this regard.

Several permanent NK cell lines have been established, and the most notable is NK-92, derived from a patient with non-Hodgkin's lymphoma expressing typical NK cell markers, with the exception of CD16 (Fc gamma receptor III). NK-92 has undergone extensive preclinical testing and exhibits superior lysis against a broad range of tumours compared with activated NK cells and lymphokine-activated killer (LAK) cells (Gong, Maki et al. 1994). Cytotoxicity of NK-92 cells against primary AML has been established (Yan, Steinherz et al. 1998). Another NK cell line, KHYG-1, has been identified as a potential contender for clinical use (Suck et al. 2005) but is reported by some as having reduced cytotoxicity so has received less attention than NK-92.

Identification of specific cancer markers are sure to help in the fight against cancer. A problem with many known cancer markers is that they are also expressed, perhaps at different levels, on healthy cells, meaning that ‘targeted’ therapies will nevertheless inevitably result in a certain amount of self-targeting.

Chimeric antigen receptors (CARs) are receptor proteins that were originally developed to give T cells the ability to target specific proteins. CARs are being investigated for their use in cancer treatment. WO 2016/201304, WO 2017/192440 and WO 2017/017184 describe the potential for using CARs on NK cells in cancer therapy.

CAR-T cells that target Mucin-1 (MUC-1) are known from Posey et al. (2016), however use of these cells is limited in that they exhibit cross-reactivity when activated and have a persistent nature when in circulation.

MUC-1 has been shown to be expressed on activated T-cells (Konowalchuk and Agrawal, Cell Immunol. 2012; 272(2):193-9) and antibodies to MUC-1 found to bind to and inhibit T-cells (Agrawal and Longenecker, Int Immunol. 2005 April; 17(4):391-9). Interaction of MUC-1 antibodies with T-cells has led to discontinuation of work on this target (Maher, et al, letter to Immunity Vol. 45, Issue 5, pp 945-946, Nov. 15, 2016).

Thus, there exists a need for alternative and preferably improved cell based therapies with greater selectivity for cancer cells.

An object of the present invention is to provide NK cells and NK cell lines that target cancer cells with high selectivity and preferably have a more cytotoxic phenotype. A further object is to provide methods for producing modified NK cells and NK cell lines, compositions containing the cells or cell lines and uses of such in the treatment of cancers. More particular embodiments aim to provide treatments for identified cancers, e.g. blood cancers, including leukemias. Specific embodiments aim at combining two or more modifications of NK cells and NK cell lines to further enhance the cytotoxicity of the modified cells.

### SUMMARY OF THE INVENTION

There are provided herein modified NK cells and NK cell lines with a more cytotoxic phenotype, and methods of making the cells and cell lines. Also provided are compositions of modified NK cells and NK cell lines, and uses of said compositions for treating cancer. Cytotoxicity in this context, as above, refers to killing of tumour cells.

The invention provides NK cells and cell lines that bind tumour-associated MUC-1 and methods of modifying NK cells and NK cell lines using, for example, genetic engineering to provide the NK cells and cell lines with the ability to bind tumour-associated MUC-1.

According to the invention, there are further provided methods of treating cancer, e.g. blood cancer, using the (modified) NK cells and lines, both generally and specifically e.g. as derivatives of KHYG-1 cells, wherein the (modified) NK cells and lines are engineered to bind tumour-associated MUC-1 and optionally in addition to lack expression of checkpoint inhibitory receptors, express TRAIL ligand variants and/or express Fc receptors.

Diseases particularly treatable according to the invention include cancers, solid cancers and blood cancers, more particularly breast cancers, ovarian cancers, colorectal cancers, multiple myelomas, leukemias and specifically acute myeloid leukemia. Tumours and cancers in humans in particular can be treated. References to tumours herein include references to neoplasms.

### Details of the Invention

Accordingly, the present invention provides a natural killer (NK) cell or NK cell line that has been genetically modified to increase its cytotoxicity.

As described in detail below in examples, NK cells and NK cell lines have been genetically modified so as to increase their cytotoxic activity against cancer.

Together, the NK cells and NK cell lines of the invention will also be referred to as the NK cells (unless the context requires otherwise).

A NK cell or NK cell line of the invention has the ability to bind a tumour-associated Mucin-1 (MUC-1) glycoform. Such glycoforms are regarded as non-natural forms of the MUC-1 antigen and are identifiable on the surface of cancers. The NK cells and cell lines hence bind aberrantly glycosylated MUC-1.

The NK cell or NK cell line may have been genetically modified to express membrane-bound moieties that bind tumour-associated MUC-1 glycoforms.

Binding of these MUC-1 glycoforms by the NK cells, illustrated in examples below, is preferably with high affinity, measured relative to binding to wildtype MUC-1 glycoforms, i.e. to non-aberrantly glycosylated MUC-1.

Binding affinity may be measured according to any suitable method known in the art. Preferably, binding affinity is measured using surface plasmon resonance, isothermal titration calorimetry or ELISA.

It is preferred that the increase in affinity of the modified NK cell for the tumour associated MUC-1 on the cancer cell is at least 10%, at least 20%, at least 50%, more preferably at least 100%, compared with its affinity for normal (non-malignant) cells expressing wildtype MUC-1.

Binding of wildtype MUC-1 by the NK cells of the invention is preferably with low affinity, measured relative to binding to cancer-related MUC-1 glycoforms, i.e. to aberrantly glycosylated MUC-1.

It is preferred that the decrease in affinity of the modified NK cell for the normal cell is at least 10%, at least 20%, at least 50%, more preferably at least 100%, compared with its affinity for cancer cells expressing aberrantly glycosylated MUC-1.

It is preferred that the increase in affinity of the modified NK cell for the cancer cell is at least 1.5-fold, at least 2-fold, at least 5-fold, at least 10-fold, at least 100-fold, more preferably at least 1000-fold greater than the affinity of the modified NK cell for a normal cell expressing wildtype MUC-1.

It is preferred that the affinity of the modified NK cell for a normal cell expressing wildtype MUC-1 is at least 1.5-fold, at least 2-fold, at least 5-fold, at least 10-fold, at least 100-fold, more preferably at least 1000-fold lower than the affinity of the modified NK for a cancer cell aberrantly glycosylated MUC-1.

Preferably, modified NK cells have high affinity for aberrantly glycosylated MUC-1, wherein ‘high affinity’ is defined as having an affinity high enough to effectively target cancer cells, but low enough to avoid targeting normal non-aberrantly glycosylated MUC-1.

Also as set out in examples below, the membrane-bound moieties are suitably modular fusion receptors that comprise a targeting sequence (commonly an antibody-derived single-chain fragment (scFv)) and usually a hinge (to overcome steric hindrance issues), a spacer, a membrane-spanning element and a signalling endodomain. Typically, the moieties are chimeric antigen receptors (CARs). Examples below use a hinge derived from IgD, other hinges are also suitable e.g. CD8a. A number of sequences are known to bind aberrantly glycosylated MUC-1 and can be used as the targeting sequence, such sequences including 5E5, SM3 and HMFG2, and are suitably incorporated in a CAR of the invention; preferably the CARs comprise the HMFG2 sequence. Nevertheless, further sequences for targeting aberrantly glycosylated MUC-1 may be identified through screening methods known in the prior art, wherein high affinity sequences (as defined above) can be used to produce MUC-1 NK-CARs according to the invention.

As such, the invention provides a NK cell or NK cell line that has been modified to express one or more CARs that bind tumour-associated MUC-1 glycoforms with increased affinity, relative to wildtype MUC-1 glycoforms.

Compared with wild type or non-aberrant MUC-1, the glycoforms associated with cancer tend to have characterising features, notably certain antigens in higher amounts. The particular glycoforms of MUC-1 targetted by the binding properties of cells and lines of the invention generally have a preponderance of shorter glycans selected from Tn, sialyl Tn (STn), T and sialyl T (ST) glycans when compared with wildtype glycoforms (Maher and Wilkie, Cancer Res, Jun. 1 2009, 69 (11), 4559-4562; DOI:10.1158/0008-5472.CAN-09-0564).

Moieties on the cells and lines of the invention may bind with higher or lower affinity to glycoforms having one or more or all of these shorter glycans. Specific sequences known to bind specific aberrantly glycosylated MUC-1 can do so with high affinity and specificity. Sequences that bind with slightly less specificity but with acceptable affinity to a plurality, and preferably all, of the listed tumour associated glycoforms are found to be of broader spectrum use in the invention. HMFG2 scFv has the advantage of targeting sialyl T antigen as well as Tn and sialyl Tn antigen; the latter two are targeted by 5E5 scFv.

An advantage of the invention is that the NK cells and lines are targetted to cancer. Whether used alone or in combination with other therapeutic components or as cells/lines with other modifications the cells can be used in cancer therapy. Data to date in vitro show cells of examples of the invention were cytotoxic.

In related T cell based therapies, MUC-1 was previously regarded as a candidate for targeting T cell action due to the specific glycosylation patterns associated with tumour-derived MUC-1. As a result, monoclonal antibodies (e.g. 5E5 and HMFG2) were developed to target these cancer-specific glycoforms. U.S. Pat. No. 8,440,798 details the monoclonal antibody 5E5. T cell chimeric antigen receptors (T-CARs) were then developed to target tumour-associated MUC-1 (Wilkie et al. 2008). Unfortunately, however, cross-reactivity with the activated T cells and the persistent nature of T cells in the circulation render the T-CAR MUC-1 therapy problematic to the extent this approach is no longer being pursued clinically.

In the present invention, using NK cells it is optional to allow for the risk of such self-targetting and provide further elements to overcome this.

Despite the risk of self targetting effects, i.e. that aberrantly glycosylated MUC-1 also be expressed on patient T cells, patient NK cells or the therapeutic NK cells themselves so that one or more of these cell types might be destroyed by the therapeutic NK cells of the invention, the therapies of the invention may be carried out with reduced durations of exposure to the therapeutic NK cells, mitigating the risks. NK cells generally do not survive for long periods in circulation, perhaps up to several weeks (though this varies), reducing the self-targetting risk. The present invention uses CAR-NK cells, not T cells which persist in the patient for months and even many months, hence the therapeutic index for the NK cells of the invention is expected to be much wider. Thus, an acceptable therapeutic effect can be achieved without significant negative side effects.

NK cells or cell lines according to the invention may also be treated or pre-treated to render them incapable of division. This results in further reduced lifetime in circulation in the patient, e.g. in comparison with T cells, further mitigating the risks above, and also with reduced or absent propensity to form tumours in a patient. These features are described also in more detail below.

NK cells or cell lines according to the invention may separately or in addition be modified so as to have reduced or absent expression of aberrantly glycosylated MUC-1. By way of example, cells and lines of the invention optionally have MUC-1 knocked down or knocked out, achievable by genetic modification e.g. using siRNA and CRISPR.

In preferred embodiments, the cell line is a derivative of the KHYG-1 cell line. In testing we have determined this cell line does not express aberrantly glycosylated MUC-1, thus avoiding self targeting effects.

NK cells and lines of the invention are for use in treating cancer in a patient. The cancer is suitably a solid cancer, e.g. breast cancer, ovarian cancer or colorectal cancer. It may be a blood cancer, especially a blood cancer selected from acute lymphocytic leukemia (ALL), acute myeloid leukemia (AML), chronic lymphocytic leukemia (CLL), chronic myeloid leukemia (CML), Hodgkin's lymphoma, non-Hodgkin's lymphoma, including T-cell lymphomas and B-cell lymphomas, asymptomatic myeloma, smoldering multiple myeloma (SMM), active myeloma and light chain myeloma; in particular it is a leukemia or multiple myeloma.

Preferably, the NK cells and NK cells lines of the invention are for use in treating cancers that are inherently resistant to NK cell-mediated cytotoxicity.

NK cell resistant cancers are well-known in the art (Pardoll, D. M. Immunity (2015) 42:605-606). Sensitivity of cancer cells to NK cell-mediated killing is determined by a number of factors. There exists a balance of positive and negative signals, largely delivered through membrane receptors on NK cells interacting with ligands on cancer cells. It is often the balance of expression of the ligands for these receptors that determines whether a cancer is sensitive or resistant to killing by NK cells (Yokoyama, W. M. Immunol Res (2005) 32:317-325).

Cancer sensitivity to NK cell-mediated cytotoxicity is generally understood as falling into one of the following categories: highly resistant, resistant, sensitive and highly sensitive. In the laboratory, cancer cells can be screened for their sensitivity to NK cell-mediated cytotoxicity through the use of cytotoxicity assays. Each category is then understood as corresponding to the percentage of cancer cells that are killed during exposure to NK cells at a specific effector:target (E:T) ratio and for a specific amount of time.

In examples of the invention, a cancer is said to be highly resistant to NK cell-mediated killing if 25% of the cancer cells are killed after incubation with NK cells for up to 15 hours at an E:T ratio of up to 5:1. Preferably, the NK cells are KHYG-1 cells.

In examples of the invention, a cancer is said to be resistant to NK cell-mediated killing if 50% of the cancer cells are killed after incubation with NK cells for up to 15 hours at an E:T ratio of up to 5:1. Preferably, the NK cells are KHYG-1 cells.

In examples of the invention, a cancer is said to be sensitive to NK cell-mediated killing if >50% of the cancer cells are killed after incubation with NK cells for up to 15 hours at an E:T ratio of up to 5:1. Preferably, the NK cells are KHYG-1 cells.

In examples of the invention, a cancer is said to be highly sensitive to NK cell-mediated killing if 75% of the cancer cells are killed after incubation with NK cells for up to 15 hours at an E:T ratio of up to 5:1. Preferably, the NK cells are KHYG-1 cells.

In preparing NK cells, the genetic modification may occur before the cell has differentiated into an NK cell. For example, pluripotent stem cells (e.g. iPSCs) can be genetically modified then differentiated to produce genetically modified CAR NK cells with increased cytotoxicity.

In certain embodiments of the invention NK cells are provided that are further modified so as to have reduced or absent checkpoint inhibitory receptor function. The combination with targeting tumour-associated MUC-1 may in particular be effective against solid tumours, especially ovarian, breast and/or colorectal. NK cells may be produced that have one or more checkpoint inhibitory receptor genes knocked out. Preferably, these receptors are specific checkpoint inhibitory receptors. Preferably still, these checkpoint inhibitory receptors are one or more or all of CD96 (TACTILE), CD152 (CTLA4), CD223 (LAG-3), CD279 (PD-1), CD328 (SIGLEC7), SIGLEC9, TIGIT and/or TIM-3. In other embodiments, NK cells are provided in which one or more inhibitory receptor signaling pathways are knocked out or exhibit reduced function—the result again being reduced or absent inhibitory receptor function. For example, signaling pathways mediated by SHP-1, SHP-2 and/or SHIP are knocked out by genetic modification of the cells.

It is preferred to reduce function of checkpoint inhibitory receptors over other inhibitory receptors, due to the expression of the former following NK cell activation. The normal or ‘classical’ inhibitory receptors, such as the majority of the KIR family, NKG2A and LIR-2, bind MHC class I and are therefore primarily involved in reducing the problem of self-targeting. Preferably, therefore, checkpoint inhibitory receptors are knocked out. Reduced or absent function of these receptors according to the invention prevents cancer cells from suppressing immune effector function (which might otherwise occur if the receptors were fully functional). Thus a key advantage of these embodiments of the invention lies in NK cells that are less susceptible to suppression of their cytotoxic activities by cancer cells; as a result they are useful in cancer treatment.

As used herein, references to inhibitory receptors generally refer to a receptor expressed on the plasma membrane of an immune effector cell, e.g. a NK cell, whereupon binding its complementary ligand resulting intracellular signals are responsible for reducing the cytotoxicity of said immune effector cell. These inhibitory receptors are expressed during both ‘resting’ and ‘activated’ states of the immune effector cell and are often associated with providing the immune system with a ‘self-tolerance’ mechanism that inhibits cytotoxic responses against cells and tissues of the body. An example is the inhibitory receptor family ‘KIR’ which are expressed on NK cells and recognize MHC class I expressed on healthy cells of the body.

Also as used herein, checkpoint inhibitory receptors are usually regarded as a subset of the inhibitory receptors above. Unlike other inhibitory receptors, however, checkpoint inhibitory receptors are expressed at higher levels during prolonged activation and cytotoxicity of an immune effector cell, e.g. a NK cell. This phenomenon is useful for dampening chronic cytotoxicity at, for example, sites of inflammation. Examples include the checkpoint inhibitory receptors PD-1, CTLA-4 and CD96, all of which are expressed on NK cells.

The invention hence also provides a NK cell that binds aberrantly glycosylated MUC-1 and also lacks a gene encoding a checkpoint inhibitory receptor selected from CD96 (TACTILE), CD152 (CTLA4), CD223 (LAG-3), CD279 (PD-1), CD328 (SIGLEC7), SIGLEC9, TIGIT and TIM-3.

Lacking a gene can refer to either a full or partial deletion, mutation or otherwise that results in no functional gene product being expressed. In embodiments, the NK cell lacks genes encoding two or more of the inhibitory receptors.

More specific embodiments comprise a NK cell lacking a gene encoding a checkpoint inhibitory receptor selected from CD96 (TACTILE), CD152 (CTLA4), CD328 (SIGLEC7) and CD279 (PD-1), especially CD96 or CD328.

In one specific embodiment, described in the examples below, a NK cell of the invention was cytotoxic against a cancer expressing a siglec ligand; for such cancers NK cells of the invention in which SIGLEC9 and/or especially SIGLEC7 is reduced or absent are preferred.

In other embodiments of the invention NK cells are provided that bind aberrantly glycosylated MUC-1 and also express a TRAIL ligand or, preferably, a mutant (variant) TRAIL ligand. The resulting NK cells exhibit increased binding to TRAIL receptors and, as a result, increased cytotoxicity against cancers, especially solid cancers, in particular ovarian, breast and colorectal cancers, and blood cancers, in particular leukemias. The NK cells with this combined activity may also be effective in reducing cancer metastases.

The TRAIL mutants/variants preferably have lower affinity (or in effect no affinity) for ‘decoy’ receptors, compared with the binding of wild type TRAIL to decoy receptors. Such decoy receptors represent a class of TRAIL receptors that bind TRAIL ligand but do not have the capacity to initiate cell death and, in some cases, act to antagonize the death signaling pathway. Mutant/variant TRAIL ligands may be prepared according to WO 2009/077857.

The mutants/variants may separately have increased affinity for TRAIL receptors, e.g. DR4 and DR5. Wildtype TRAIL is typically known to have a K_{D} of >2 nM for DR4, >5 nM for DR5 and >20 nM for the decoy receptor DcR1 (WO 2009/077857; measured by surface plasmon resonance), or around 50 to 100 nM for DR4, 1 to 10 nM for DR5 and 175 to 225 nM for DcR1 (Truneh, A. et al. 2000; measured by isothermal titration calorimetry and ELISA). Therefore, an increased affinity for DR4 is suitably defined as a K_{D} of <2 nM or <50 nM, respectively, whereas an increased affinity for DR5 is suitably defined as a K_{D} of <5 nM or <1 nM, respectively. A reduced affinity for decoy receptor DcR1 is suitably defined as a K_{D} of >50 nM or >225 nM, respectively. In any case, an increase or decrease in affinity exhibited by the TRAIL variant/mutant is relative to a baseline affinity exhibited by wildtype TRAIL. The affinity is preferably increased at least 10%, at least 25%, at least 50%, more preferably at least 100% compared with that exhibited by wildtype TRAIL.

The TRAIL variant preferably has an increased affinity for DR5 as compared with its affinity for DR4, DcR1 and DcR2. Preferably, the affinity is at least 1.5-fold, 2-fold, 5-fold, 10-fold, 100-fold, or even 1,000-fold or greater for DR5 than for one or more of DR4, DcR1 and DcR2. More preferably, the affinity is at least 1.5-fold, 2-fold, 5-fold, 10-fold, 100-fold, or even 1,000-fold or greater for DR5 than for at least two, and preferably all, of DR4, DcR1 and DcR2.

The TRAIL variant preferably has an increased affinity for one or both of DR4 and DR5 as compared with its affinity for wildtype TRAIL. Preferably, the affinity is at least 1.5-fold, 2-fold, 5-fold, 10-fold, 100-fold, or even 1,000-fold or greater for DR4 and/or DR5 than for wildtype TRAIL.

High affinity TRAIL variants have increased affinity for TRAIL death receptors as defined above.

Further specific embodiments comprise a NK cell expressing a mutant TRAIL ligand that has reduced or no affinity for TRAIL decoy receptors. Further specific embodiments comprise a NK cell expressing a mutant TRAIL ligand that has reduced or no affinity for TRAIL decoy receptors and increased affinity for DR4 and/or DR5.

Treatment of a cancer using modified NK cells expressing TRAIL or a TRAIL variant is optionally enhanced by administering to a patient an agent capable of upregulating expression of TRAIL death receptors on cancer cells. This agent may be administered prior to, in combination with or subsequently to administration of the modified NK cells. It is preferable, however, that the agent is administered prior to administering the modified NK cells. The agent upregulates expression of DR5 on cancer cells. The agent may optionally be a chemotherapeutic medication, e.g. a proteasome inhibitor, one of which is Bortezomib, and administered in a low dose capable of upregulating DR5 expression on the cancer. Other examples of DR5-inducing agents include Gefitinib, Piperlongumine, Doxorubicin, Alpha-tocopheryl succinate and HDAC inhibitors.

Binding to aberrantly glycosylated MUC-1 is preferably achieved using a CAR. The CARs used in the cells or lines of the invention may comprise or be linked to one or more NK cell costimulatory domains, e.g. CD28, CD134/OX40, 4-1BB/CD137, CD3zeta/CD247, DAP12 or DAP10. Binding of the variant to its receptor on a target cell thus promotes apoptotic signals within the target cell, as well as stimulating cytotoxic signals in the NK cell. In examples below, the CAR comprised CD28, OX40 and CD3zeta.

According to further preferred embodiments of the invention, NK cells are provided that express a CAR that binds aberrantly glycosylated MUC-1, have reduced checkpoint inhibitory receptor function and also express a mutant TRAIL ligand, as described in more detail above in relation to these respective NK cell modifications.

Optional features of the invention include providing further modifications to the NK cells and NK cell lines described above, wherein, for example, a Fc receptor (which can be CD16, CD32 or CD64, including subtypes and derivatives) is expressed on the surface of the cell. In use, these cells can show increased recognition of antibody-coated cancer cells and improved activation of the cytotoxic response.

Further optional features of the invention include adapting the modified NK cells and NK cell lines to better home to specific target regions of the body. NK cells of the invention may be targeted to specific cancer cell locations. In preferred embodiments for treatment of blood cancers, NK effectors of the invention are adapted to home to bone marrow. Specific NK cells are modified by fucosylation and/or sialylation to home to bone marrow. This may be achieved by genetically modifying the NK cells to express the appropriate fucosyltransferase and/or sialyltransferase, respectively. Increased homing of NK effector cells to tumour sites may also be made possible by disruption of the tumour vasculature, e.g. by metronomic chemotherapy, or by using drugs targeting angiogenesis (Melero et al, 2014) to normalize NK cell infiltration via cancer blood vessels.

Yet another optional feature of the invention is to provide modified NK cells and NK cell lines with an increased intrinsic capacity for rapid growth and proliferation in culture. This can be achieved, for example, by transfecting the cells to overexpress growth-inducing cytokines IL-2 and IL-15. Moreover, this optional alteration provides a cost-effective alternative to replenishing the growth medium with cytokines on a continuous basis. These cells can be used as intermediates to prepare significant quantities of cells for subsequent processing (e.g. to reduce their divisional capacity) prior to therapeutic application (which as noted can include treatment such as irradiation to prevent division in the patient).

The invention further provides a method of making a modified NK cell or NK cell line, comprising genetically modifying the cell or cell line as described herein so as to express a CAR that binds aberrantly glycosylated MUC-1.

Modified NK cells, NK cell lines and compositions thereof described herein, above and below, are suitable for treatment of cancer, in particular cancer in humans, e.g. for treatment of cancers of blood cells or solid cancers. The NK cells and derivatives are preferably human NK cells. For human therapy, human NK cells are preferably used. The invention also provides methods of treating cancer in humans comprising administering an effective amount of the cells or lines or compositions.

Various routes of administration will be known to the skilled person to deliver active agents and combinations thereof to a patient in need. Embodiments of the invention are for blood cancer treatment. Administration of the modified NK cells and/or NK cell lines can be systemic or localized, such as for example via the intraperitoneal route.

In other embodiments, active agent is administered more directly. Thus administration can be directly intratumoural, suitable especially for solid tumours.

NK cells in general are believed suitable for the methods, uses and compositions of the invention. As per cells used in certain examples herein, the NK cell can be a NK cell obtained from a cancer cell line. Advantageously, a NK cell, preferably treated to reduce its tumourigenicity, for example by rendering it mortal and/or incapable of dividing, can be obtained from a blood cancer cell line and used in methods of the invention to treat blood cancer.

To render a cancer-derived NK cell more acceptable for therapeutic use, it is generally treated or pre-treated in some way to reduce or remove its propensity to form tumours in the patient. Specific modified NK cell lines used in examples are safe because they have been rendered incapable of division; they are irradiated and retain their killing ability but die within about 3-4 days. Specific cells and cell lines are hence incapable of proliferation, e.g. as a result of irradiation. Treatments of potential NK cells for use in the methods herein include irradiation to prevent them from dividing and forming a tumour in vivo and genetic modification to reduce tumourigenicity, e.g. to insert a sequence encoding a suicide gene that can be activated to prevent the cells from dividing and forming a tumour in vivo. Suicide genes can be turned on by exogenous, e.g. circulating, agents that then cause cell death in those cells expressing the gene. A further alternative is the use of monoclonal antibodies targeting specific NK cells of the therapy. CD52, for example, is expressed on KHYG-1 cells and binding of monoclonal antibodies to this marker can result in antibody-dependent cell-mediated cytotoxicity (ADCC) and KHYG-1 cell death.

As discussed in an article published by Suck et al, 2006, cancer-derived NK cells and cell lines are easily irradiated using irradiators such as the Gammacell 3000 Elan. A source of Cesium-137 is used to control the dosing of radiation and a dose-response curve between, for example, 1 Gy and 50 Gy can be used to determine the optimal dose for eliminating the proliferative capacity of the cells, whilst maintaining the benefits of increased cytotoxicity. This is achieved by assaying the cells for cytotoxicity after each dose of radiation has been administered.

There are significant benefits of using an irradiated NK cell line for adoptive cellular immunotherapy over the well-established autologous or MHC-matched T cell approach. Firstly, the use of a NK cell line with a highly proliferative nature means expansion of modified NK cell lines can be achieved more easily and on a commercial level. Irradiation of the modified NK cell line can then be carried out prior to administration of the cells to the patient. These irradiated cells, which retain their useful cytotoxicity, have a limited life span and, unlike modified T cells, will not circulate for long periods of time causing persistent side-effects.

Additionally, the use of allogeneic modified NK cells and NK cell lines means that MHC class I expressing cells in the patient are unable to inhibit NK cytotoxic responses in the same way as they can to autologous NK cytotoxic responses. The use of allogeneic NK cells and cell lines for cancer cell killing benefits from the previously mentioned GVL effect and, unlike for T cells, allogeneic NK cells and cell lines do not stimulate the onset of GVHD, making them a preferred option for the treatment of cancer via adoptive cellular immunotherapy.

In a further aspect of the invention, there is provided a method of assessing the sensitivity of a cancer to NK cell-mediated cytotoxicity, before determining whether to proceed with treating the cancer with the MUC-1 NK-CARs described above and below.

Accordingly, there is provided a method comprising the steps of (1) isolating one or more cancer cells from a patient, (2) determining whether the one or more cancer cells is NK cell resistant by measuring the % specific cytotoxicity of NK cells to the one or more cancer cells, and (3) making a decision as to whether or not to treat the cancer based on the findings of step (2), characterised in that treatment of the cancer is carried out if the one or more cancer cells is NK cell resistant.

Preferably, NK cell resistance is defined as a % specific cytotoxicity of 50% or below.

Preferably, MUC-1 NK-CARs of the present invention are used to treat the patient's cancer that is inherently resistant to NK cell-mediated cytotoxicity.

### EMBODIMENTS

According to the present invention, the following embodiments are provided:

1. A natural killer (NK) cell or NK cell line that has been modified so as to bind tumour-associated Mucin-1 (MUC-1) glycoforms.

2. A NK cell or NK cell line according to embodiment 1, wherein the NK cell or NK cell line has been genetically modified to express membrane-bound moieties that bind tumour-associated MUC-1 glycoforms with high affinity, relative to wildtype MUC-1 glycoforms.

3. A NK cell or NK cell line according to embodiment 2, wherein the membrane-bound moieties are chimeric antigen receptors (CARs).

4. A NK cell or NK cell line according to embodiment 3, wherein the CARs comprise the HMFG2 sequence.

5. A NK cell or NK cell line according to any previous embodiment, wherein the tumour-associated MUC-1 glycoforms comprise a preponderance of shorter glycans selected from Tn, sialyl Tn (STn), T, and sialyl T (ST) glycans, when compared with wildtype glycoforms.

6. A NK cell or cell line according to any previous embodiment, having reduced or absent propensity to form tumours in a patient.

7. A NK cell or NK cell line according to embodiment 6, which has been rendered incapable of division, e.g. by irradiation.

8. A NK cell or cell line according to any previous embodiment, modified so as to have reduced or absent expression of aberrantly glycosylated MUC-1.

9. A NK cell or NK cell line according to any previous embodiment, further modified to express a mutant TRAIL ligand with high affinity for one or more TRAIL death receptors, when compared to wildtype TRAIL.

10. A NK cell or NK cell line according to embodiment 9, wherein the mutant TRAIL ligand comprises the D269H/E195R mutation.

11. A NK cell or NK cell line according to any previous embodiment, further modified to remove function of one or more checkpoint inhibitory receptors.

12. A NK cell or NK cell line according to any previous embodiment, wherein the cell line is a derivative of the KHYG-1 cell line.

13 A NK cell or NK cell line according to any previous embodiment for use in treating cancer in a patient.

14. A NK cell or NK cell line for use according to embodiment 13, wherein the cancer is a solid cancer, e.g. selected from breast cancer, ovarian cancer and colorectal cancer.

15. A NK cell or NK cell line for use according to embodiment 14, wherein the cancer is a blood cancer, e.g. selected from acute lymphocytic leukemia (ALL), acute myeloid leukemia (AML), chronic lymphocytic leukemia (CLL), chronic myeloid leukemia (CML), Hodgkin's lymphoma, non-Hodgkin's lymphoma, including T-cell lymphomas and B-cell lymphomas, asymptomatic myeloma, smoldering multiple myeloma (SMM), active myeloma and light chain myeloma.

### EXAMPLES

The present invention is now described in more and specific details in relation to the production of NK cell line KHYG-1 derivatives, modified to exhibit more cytotoxic activity through an ability to bind aberrantly glycosylated MUC-1.

The invention is now illustrated in specific embodiments with reference to the accompanying drawings in which:

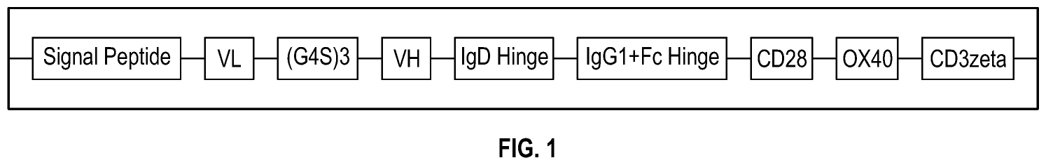

FIG. 1 shows the gene sequence organisation of a MUC-1 chimeric antigen receptor (CAR);

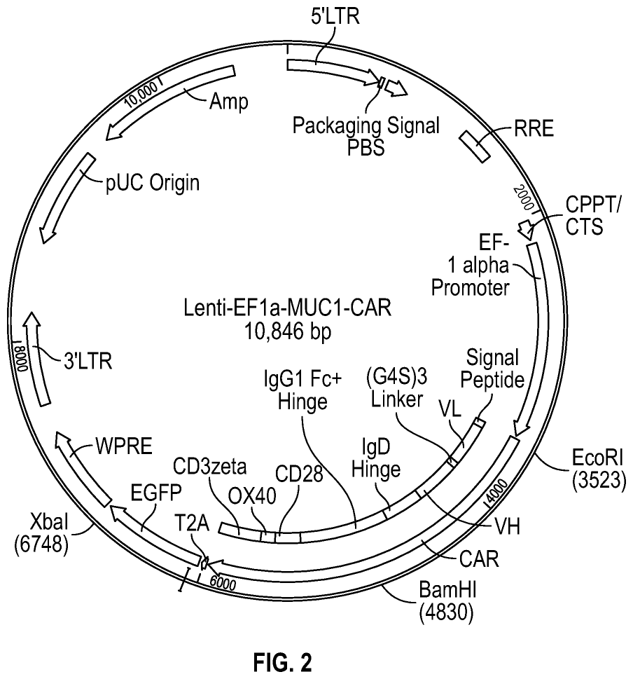

FIG. 2 shows the vector map of Lenti-EF1a-MUC1-CAR used to express the CAR in KHYG-1 cells;

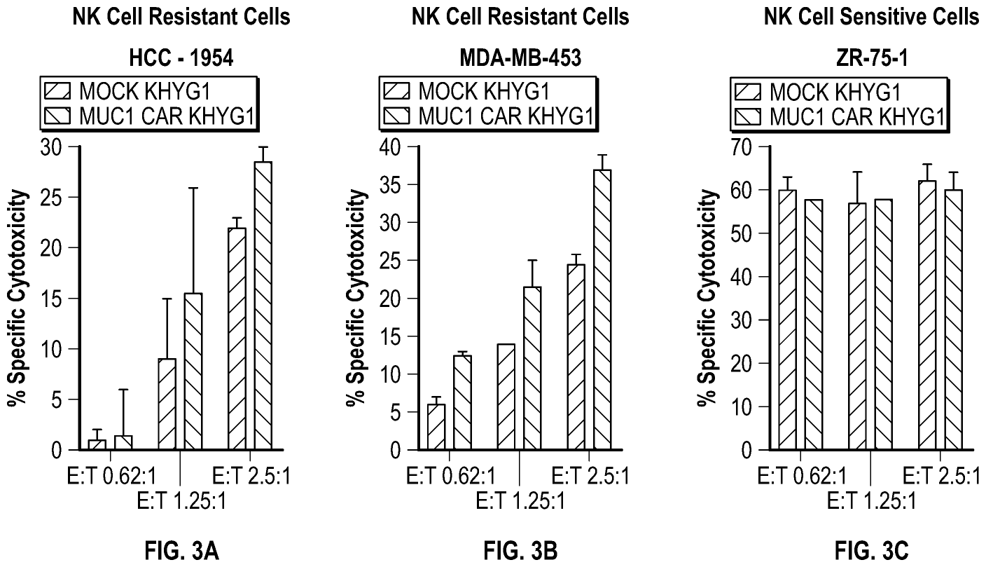

FIGS. 3a 3b and 3c show the killing of different breast cancer cells using MUC-1 CAR-NK cells; and

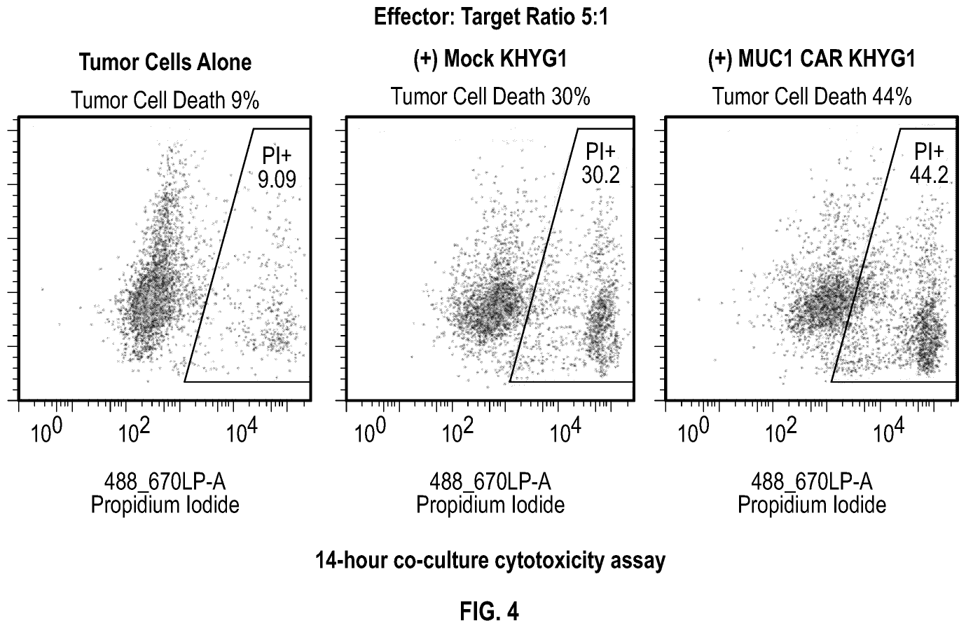

FIG. 4 shows the killing of MDA-MB-453 breast cancer cells using MUC-1 CAR-NK cells at an effector:target ratio of 5:1.

DNA, RNA and amino acid sequences are referred to below, in which:
SEQ ID NO: 1 is the MUC-1 CAR DNA sequence;
SEQ ID NO: 2 is the MUC-1 CAR peptide sequence; and
SEQ ID NO: 3 is the 24-mer peptide staining sequence used for MUC-1 binding.

SEQ ID NO: 1
ATGTGGCAACTGCTGCTGCCTACAGCTCTGCTGCTTCTGGTGTCCGCCGA
TATCGTGGTCACACAAGAGAGCGCCCTGACCACCTCTCCTGGCGAAACAG
TGACCCTGACCTGCAGATCTTCTACAGGCGCCGTGACCACAAGCAACTAC
GCCAACTGGGTGCAAGAGAAGCCCGATCACCTGTTCACAGGCCTGATCGG
CGGCACAAACAATAGAGCACCTGGCGTGCCAGCCAGATTCAGCGGATCTC
TGATCGGAGACAAGGCCGCACTGACAATCACAGGCGCCCAGACAGAGGAC
GAGGCCATCTACTTTTGCGCCCTGTGGTACAGCAACCACTGGGTTTTCGG
CGGAGGCACCAAGCTGACAGTGCTGGGATCTGAAGGTGGCGGAGGATCTG
GCGGAGGTGGAAGCGGAGGCGGAGGTTCTGAAGTTCAGCTGCAACAATCT
GGCGGCGGACTGGTTCAACCTGGCGGCTCTATGAAGCTGAGCTGTGTGGC
CAGCGGCTTCACCTTCAGCAACTACTGGATGAACTGGGTCCGACAGAGCC
CCGAGAAAGGCCTGGAATGGGTTGCCGAGATCAGACTGAAGTCCAACAAT
TACGCCACACACTACGCCGAGAGCGTGAAGGGCAGATTCACCATCAGCCG
GGACGACAGCAAGAGCAGCGTGTACCTCCAGATGAACAACCTGAGAGCCG
AGGACACCGGCATCTACTACTGCACCTTCGGCAACAGCTTCGCCTATTGG
GGCCAGGGAACCACCGTGACCGTGTCCAGCACCTTCACCTGTTTTGTCGT
GGGCAGCGACCTGAAGGATGCCCACCTGACATGGGAAGTCGCCGGCAAAG
TTCCTACCGGTGGCGTGGAAGAAGGCCTGCTGGAAAGACACAGCAACGGC
AGCCAGAGCCAGCACAGCAGACTGACACTGCCTAGAAGCCTGTGGAATGC
CGGCACCAGCGTGACCTGCACACTGAATCATCCTAGCCTGCCTCCACAGA
GACTGATGGCCCTGAGAGAACCTGCTGCTCAGGCCCCTGTGAAGCTGTCC
CTGAATCTGCTCGCCAGCAGCGATCCTCCTGAAGCCGCCAATGTGAACCA
CAAGCCTAGCAACACCAAGGTGGACAAGAAGGTGGAACCCAAGAGCTGCG
ACAAGACCCACACCTGTCCTCCATGTCCTGCTCCAGAACTGCTCGGCGGA
CCTTCCGTGTTCCTGTTTCCTCCAAAGCCTAAGGACACCCTGATGATCAG
CAGAACCCCTGAAGTGACCTGCGTGGTGGTGGATGTGTCCCACGAGGATC
CCGAAGTGAAGTTCAATTGGTACGTGGACGGCGTCGAGGTGCACAACGCC
AAGACAAAGCCCAGAGAGGAACAGTACAACAGCACCTACAGAGTGGTGTC
CGTGCTGACCGTGCTGCACCAGGATTGGCTGAACGGCAAAGAGTACAAGT
GCAAGGTGTCCAACAAGGCCCTGCCTGCTCCTATCGAGAAAACCATCAGC
AAGGCCAAGGGCCAGCCTAGAGAACCCCAGGTGTACACACTGCCTCCAAG
CAGAGATGAGCTGACCAAGAACCAGGTGTCCCTGACATGCCTGGTCAAGG
GCTTCTACCCCTCCGATATCGCCGTGGAATGGGAGAGCAATGGACAGCCC
GAGAACAACTACAAGACAACCCCTCCTGTGCTGGACTCCGACGGCTCATT
CTTCCTGTACAGCAAACTGACCGTGGACAAGTCCAGATGGCAGCAGGGCA
ACGTGTTCTCCTGCAGCGTGATGCACGAGGCCCTGCACTTTTGGGTGCTC
GTGGTTGTTGGCGGAGTGCTGGCCTGTTACAGCCTGCTGGTTACCGTGGC
CTTCATCATCTTTTGGGTCCGAAGCAAGCGGAGCCGGCTGCTGCACAGCG
ACTACATGAACATGACCCCTAGACGGCCCGGACCTACCAGAAAGCACTAC
CAGCCTTACGCTCCTCCTAGAGACTTCGCCGCCTACAGAAGCAGACGGGA
CCAAAGACTGCCTCCTGACGCTCACAAACCTCCAGGCGGCGGAAGCTTCA
GGACCCCTATCCAAGAAGAACAGGCTGACGCCCACAGCACCCTGGCCAAG
ATCCGCGTGAAGTTCTCCAGATCCGCCGACGCTCCTGCCTATCAGCAGGG
ACAGAACCAGCTGTACAACGAGCTGAACCTGGGGAGAAGAGAAGAGTACG
ACGTGCTGGATAAGCGGAGAGGCAGAGATCCTGAGATGGGCGGAAAGCCC
CAGCGGAGAAAGAATCCTCAAGAGGGCCTGTATAATGAGCTGCAGAAAGA
CAAGATGGCCGAGGCCTACAGCGAGATCGGAATGAAGGGCGAGCGCAGAA
GAGGCAAGGGACACGATGGACTGTACCAGGGACTGAGCACCGCCACCAAG
GATACCTATGACGCCCTGCACATGCAGGCTCTGCCTCCA
SEQ ID NO: 2
MWQLLLPTALLLLVSADIVVTQESALTTSPGETVTLTCRSSTGAVTTSNY
ANWVQEKPDHLFTGLIGGTNNRAPGVPARFSGSLIGDKAALTITGAQTED
EAIYFCALWYSNHWVFGGGTKLTVLGSEGGGGSGGGGSGGGGSEVQLQQS
GGGLVQPGGSMKLSCVASGFTFSNYWMNWVRQSPEKGLEWVAEIRLKSNN
YATHYAESVKGRFTISRDDSKSSVYLQMNNLRAEDTGIYYCTFGNSFAYW
GQGTTVTVSSTFTCFVVGSDLKDAHLTWEVAGKVPTGGVEEGLLERHSNG
SQSQHSRLTLPRSLWNAGTSVTCTLNHPSLPPQRLMALREPAAQAPVKLS
LNLLASSDPPEAANVNHKPSNTKVDI(KVEPKSCDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHFWV
LVVVGGVLACYSLLVTVAFIIFWVRSKRSRLLHSDYMNMTPRRPGPTRKH
YQPYAPPRDFAAYRSRRDQRLPPDAHKPPGGGSFRTPIQEEQADAHSTLA
KIRVKFSRSADAPAYQQGQNQLYNELNLGRREEYDVLDKRRGRDPEMGGK
PQRRKNPQEGLYNELQKDKMAEAYSEIGMKGERRRGKGHDGLYQGLSTAT
KDTYDALHMQALPP
SEQ ID NO: 3
TAPPAHGVTSAPDTRPAPGSTAPP

The expected molecular weight of the MUC1 chimeric antigen receptor is 813 amino acids=89.6 kDa, within which the sequence of single chain variable fragment and activating domains of the MUC1 CAR are as follows:

| Sequence | | | |
|---|---|---|---|
| position | Sequence Type | Base Pair length | |
| 1 | Signal peptide | 48 | bp |
| 2 | VL (Variable region light chain) | 336 | bp |
| 3 | (G4S)3 Linker sequence | 45 | bp |
| 4 | VH (Variable region heavy chain) | 351 | bp |
| 5 | IgD Hinge | 309 | bp |
| 6 | IgG1 Fc+ Hinge | 699 | bp |
| 7 | CD28 | 204 | bp |
| 8 | OX40 | 111 | bp |
| 9 | CD3zeta | 336 | bp |

For related examples of knockout/knockdown of inhibitory receptor function and knock in of mutant TRAIL we refer to WO 2017/017184 the contents of which are incorporated herein by reference.

### Example 1—Lentiviral Plasmids Encoding MUC-1-CARs

DNA nucleotides encoding a single chain variable fragment (scFv) derived from an HMFG2 clone that recognises tumour-associated antigen MUC-1 (SEQ ID NO: 1), were cloned into pCDCAR1-GFP vectors. This scFv sequence was followed by an additional immunoglobulin-based hinge region to overcome the stearic hindrance of MUC-1. The hinge regions were followed by the co-stimulatory activating domains of CD28, OX40 and CD3zeta, in order to provide activating signals for NK cell cytotoxicity, thereby triggering cytolysis of MUC-1 positive tumour cells. Following the chimeric antigen receptor (CAR) protein (SEQ ID NO: 2), the selection marker Enhanced Green Fluorescent Protein (EGFP) was positioned downstream of the T2A sequence, separating the CAR and the EGFP. The sequence was cloned between EcoRI and XbaI restriction sites of the pCDCAR1-GFP vector (see FIGS. 1 and 2). 150 μl vials of competent E. coli bacteria were thawed on ice. In the meantime, unlabelled tubes were chilled on ice. Once the last crystal had melted, 50 μl of the cells were promptly transferred to each chilled tube, along with 1 μl of the appropriate diluted plasmid solution (5 ng/μl). The tubes were then left on ice for 30 minutes. The cells were heat-shocked by submerging the tubes in a water bath set at 42° C. for 30 seconds and then allowing them to cool back on the ice for 5 minutes. 950 μl of room temperature LB broth was added to each tube and then the tubes were flicked gently to make a uniform mixture. The tubes were placed on a shaker (250 rpm) in the incubator (37° C.) for 1 hour. Next, 50 μl of each plasmid solution was added into its corresponding agar plate containing ampicillin. The plates were stored in the incubator upside down overnight to allow bacteria containing the plasmid to grow out and form single colonies (14-16 hrs). A colony was selected from one of the plates. Using an autoclaved pipette tip, a colony was taken up and smeared against the inner surface of the corresponding culture tube. The tubes were then left on a shaker (125 rpm) in the incubator (37° C.) overnight for a maximum of 16 hours. The plasmid DNA was isolated according to the manufacturer's instructions. The plasmid was verified by restriction digestion with restriction enzymes. The bacterial culture (2.5 ml) was added to 247.5 ml LB broth containing Ampicillin. The 250 ml bacterial solution was then split into 50 ml tubes and stored at −20° C.

The labelled 50 ml tubes containing the bacterial cell pellets were removed from the −20° C. freezer and allowed to thaw on the bench for a few minutes. The first cell pellet was vortexed and re-suspended in 12 ml of re-suspension buffer containing RNase A. 12 ml of Lysis Buffer was added to the suspensions. The suspensions were inverted for mixing and then incubated at room temperature for 5 minutes. 12 ml of neutralization buffer was added to each suspension, followed by inverting the tubes until the sample turned from blue to colourless. The crude lysates were then incubated on ice for 5 minutes. A column filter was inserted into NucleoBond Xtra columns, before applying 15 ml of Equilibration buffer onto the rim of each column filter to ensure moistening of the entire column and its circumference. The column was emptied by gravity flow into a basin under a column rack. The lysates were poured into their designated column filters. The column filters were washed with 5 ml Filter Wash Buffer, making sure to apply the buffer to the funnel shaped rim of the filter as mentioned above. The column was then emptied by gravity flow. The column filter was removed and discarded. The NucleoBond Xtra Column was washed with 35 ml of Wash Buffer, before allowing the column to empty by gravity flow. For washes of the column, the buffer was gently pipetted directly into the centre as opposed to on the rim. This wash step was then repeated. A 15 ml tube was positioned underneath each of the columns to collect elute. The plasmid DNA was eluted from the columns with 5 ml of Elution Buffer. 3.5 ml of room-temperature isopropanol was pipetted into each 15 ml tube, in order to precipitate the eluted plasmid DNA. The tubes were then vortexed thoroughly. The tubes were centrifuged at 4500 rpm for 40 minutes at 4° C., before carefully aspirating the supernatants. 2 ml of endotoxin-free room-temperature 70% ethanol was added to each pellet. The tubes were then centrifuged at 4500×g for 10 minutes at room temperature. The ethanol from each tube was removed using a pipette and left to dry under a hood at room temperature until the pellets turned from being slightly opaque to a more translucent glassy appearance. The DNA concentration was then measured using a Nanodrop spectrophotometer, and the DNA pellets were dissolved in 400 μl of H2O-EF.

### Example 2—Nucleofection of MUC-1 CAR Plasmids into KHYG-1 Cells

KHYG-1 cells were passaged at 1:1 (5 ml cells+5 ml culture media) the day before nucleofection in T25 flasks, while the cells were in the logarithmic growth phase. The Lonza Nucleofection kit T (Cat: VCA-1002) was used; one nucleofection sample contained 100 μl nucleofection solution (standard cuvette) and 2×10⁶ cells. The nucleofection solution contained 18 μl Supplement and 82 μl Nulceofector solution (per sample) prepared fresh and incubated at 37° C. for 10 minutes.

Solution T was warmed to room temperature. A fresh 12 ml aliquot of culture medium (CM) was prepared containing 2.4 ml FBS, 9.6 ml RPMI 1640 and supplements 6 μl IL-2 (RPMI1640+20% FBS+500 IU/ml IL-2) at 37° C. in a 15 ml tube (no antibiotics). 4 ml of CM was aliquoted into T25 flasks, and plates were pre-incubated in a humidified 37° C. incubator for 20 minutes. 10 ml cell culture in 15 ml tubes was taken and the cells were counted to determine the cell density. A 2×10⁶ sample was dispensed into 15 ml tubes and centrifuged at 1000 RPM for 5 min (acc. 9/decc.7).

The supernatant completely discarded so that no residual medium covered the cell pellet. The cell pellet was re-suspended in 100 μl room temperature Nucleofector Solution (see above) to a final concentration of 2×10⁶ cells/100 μl. Storing the cell suspension longer than 5 min in Nucleofector Solution was avoided, as this is known to reduce cell viability and gene transfer efficiency. 0.2-2 μg Lenti-EF1a-MUC1-CAR Plasmid DNA was added to each tube. The sample was then transferred into an Amaxa certified cuvette, making sure that the sample covered the bottom of the cuvette, in order to avoid air bubbles while pipetting. The cuvette was closed with a blue cap and Nucleofector program A-024 was selected. The cuvette was inserted into the cuvette holder, before pressing the “x” button to start the program (NB: program U-001 can also be used). To avoid damage to the cells, the samples were removed from the cuvette immediately after the program had finished (display showing “OK”). The pre-warmed culture medium was added to the cuvette and the sample transferred into one T25 flask. The “X” button was pressed to reset the Nucleofector. The cells were incubated in a humidified 37° C./5% CO₂ incubator for 48 hours. 2-4 ml fresh media (RPMI1640+10% FBS+100 IU/ml IL-2) was added and the cells were incubated for another 24-96 hours to establish optimal protein expression (NB: the cell culture was monitored after every 24 hours using a microscope to check the status and condition of the cells). Flow cytometric analysis was performed between 72 and 240 hours post-nucleofection.

The MUC-1 CAR plasmid had EGFP as a selection marker. Therefore, KHYG-1 cells expressing MUC-1 CARs can be determined by estimating the number of GFP-positive KHYG-1 cells.

0.5 ml cell suspension (1×10⁵ cells) was centrifuged at 1200 rpm for 5 mins. The supernatant was discarded, before adding 1 ml FACS buffer and centrifuging again at 1200 rpm for 5 mins. The cells were re-suspended in a final volume of 200 μl FACS buffer.

The cells were then analysed by flow cytometry using a 488 nm laser and detection filter of 530/30 on a FAGS Canto A—presence of GFP-positive cells confirmed successful transfection.

### Example 3—Killing of Blood Cancer Cells by MUC-1 CAR-KHYG-1 Cells

NK cell cytotoxicity was measured using U266 and RPMI-8226 target multiple myeloma cells (both FAGS screened in advance, confirming MUC-1 expression on the targets; RPMI-8226 also expresses siglec ligand) at effector:target (E:T) ratios of 0.125:1, 0.25:1, 0.5:1, 1:1 and 2:1 in a 14 hour assay with 40,000 target tumour cells per 10001, plated in a 96 well flat bottom plate. Mock transfected KHYG-1 cells or MUC-1-CAR transfected KHYG-1 cells were centrifuged, and the supernatant was discarded. Cells were re-suspended in 1 ml fresh media (RPMI1640+10% FBS+100 IU/ml IL-2) and counted. KHYG-1 cells were then brought to a final concentration of 800,000 cells/100 μl and combined with the tumour cells in a final volume of 200 μl. The IL-2 concentration in the final reaction was 50 IU/ml IL-2. The plates were then incubated at 37° C. and 5% CO₂ for 14 hours.

NK cell-induced target cell death was determined by flow cytometry. FACS tubes were pre-filled with 200 μl FACS buffer using Eppendorf repeater units, before centrifuging at 2000 RPM for 3 minutes. The supernatant was discarded by inverting and then blotting on dry clean paper. Cells were re-suspended in the tubes using a vortex. 4 μl of diluted CD2 BV421 antibody was added and incubated for 20 mins on ice and in the dark. 200 μl FACS buffer was added using an Eppendorf repeater unit, before spinning at 2000 RPM for 3 minutes and then discarding the supernatant by inverting and then blotting on clean dry paper. The cells were then re-suspended in the tubes using a vortex. 200 μl FACS buffer was added to each of 30 tubes with Eppendorf repeater units. 2 μl of Propidium Iodide was added to each tube, before waiting 2-3 mins and analysing each tube using flow cytometry.

CD2 expression on KHYG1 cells was measured using a 405 nm laser and detector 450/50, while Propidium Iodide was measured by exciting the cells with a 488 nm laser and detection filter of 585/42 on a FACS Canto II.

Results confirmed the MUC-1 CAR KHYG-1 cells demonstrated increased cytotoxicity over KHYG-1 cells not expressing the CAR against both U266 and RPMI8226 targets.

### Example 4—Killing of Breast Cancer Cells by MUC-1 CAR-KHYG-1 Cells

Expression of MUC-1 on Breast Cancer Cells

MUC-1 expression on breast cancer cell lines HCC-1954, MDA-MB-1954 and ZR-75-1 was analysed using flowcytometry. Briefly, cells were stained with anti-MUC1 (Clone: HMFG2)—AF647 (Cat: BD 566590) for 25 minutes on ice, and subsequently MUC-1 expression was measured on a FACS Canto II using 633/660/20. Expression analysis revealed that all the breast cancer cell lines had expression of MUC-1 on the cell surface, albeit at different levels. MUC-1 therefore represented a suitable candidate target antigen for the MUC-1 NK-CARs of the invention.

Transfection of NK Cells with MUC-1 CARs

mRNA for the MUC-1 CAR was synthesized using in vitro transcription (IVT) at Trilink Biotech. The EGFP sequence was excluded from the mRNA construct to reduce the mRNA size without compromising CAR activity.

KHYG-1 cells were electroporated with 12.5 μg of MUC-1 CAR expressing mRNA. KHYG-1 cells were mock electroporated for use as a control. Both samples were incubated for 24 hours to allow for protein expression of the MUC-1 CAR construct. After 24 hours, cells were stained with a 24-mer peptide sequence (TAPPAHGVTSAPDTRPAPGSTAPP-OH) and conjugated to 5(6)Carboxy-fluorescein (JPT Peptide Solutions) for 30 minutes; this peptide sequence binds MUC-1 CAR specifically. After electroporation, 86% KHYG-1 cells were positive for MUC-1 CAR expression. Mock electroporated KHYG-1 cells showed <1% MUC-1 CAR+ cells 24 hours post electroporation. Thus, successful expression of the MUC-1 CAR was demonstrated in NK cells.

Cytotoxicity of MUC-1 NK-CARs Against Breast Cancer Cells

NK cell cytotoxicity assays were performed in 96 well flat bottom plates in 200 μl final volume. After 14 hour co-culture, cells were harvested and stained with CD2-BV-421 antibody (BD Biosciences) for 30 minutes to distinguish the CD2-negative breast cancer cells from CD2-negative KHYG-1 cells. Cell death was analysed by addition of 1.5 μl of 100 μg/ml Propidium Iodide solution and incubating the cells on ice for 2 minutes before flowcytometry analysis.

Upon co-culture with MUC-1 NK-CARs with a panel of breast cancer cell lines, it was observed that the MUC-1 NK-CARs were more cytotoxic towards NK cell resistant breast cancer cell lines (HCC-1954 and MDA-MB-453) than mock electroporated KHYG-1. This observation was valid at multiple effector:target (E:T) ratios (see FIGS. 3a, 3b and 4).

There was no difference in cytotoxicity between the MUC-1 NK-CARs of the invention and the control cells (mock electroporated KHYG-1) against ZR-75-1 cells. This observation can be explained as the ZR-75-1 cells are already sensitive to NK cell mediated killing, as can be seen in FIG. 3c. Data is representative of a 14-hour cytotoxicity co-culture assay. NK cell induced tumour lysis was determined by flowcytometry using Propidium Iodide, after gating for the CD2-negative breast cancer cells.

Thus, it is demonstrated that the MUC-1 NK-CARs of the invention are particularly effective at killing a range of cancer types, especially in cases where the cancer is inherently resistant to NK cell-mediated cytotoxicity.

The invention thus provides NK cells and cell lines, and production thereof, for use in blood cancer therapy.

1-17. (canceled)

18. A natural killer (NK) cell or NK cell line modified to express a chimeric antigen receptor (CAR) that binds a tumour-associated Mucin-1 (MUC-1) glycoform.

19. The NK cell or NK cell line of claim 18, wherein the CAR binds the tumour-associated MUC-1 glycoform with increased affinity relative to wildtype MUC-1 glycoforms.

20. The NK cell or NK cell line of claim 19, wherein the increased affinity is at least 10%.

21. The NK cell or NK cell line of claim 19, wherein the increased affinity is at least 50%.

22. The NK cell or NK cell line of claim 18, wherein the CAR comprises a short-chain variable fragment (scFv) derived from 5E5, SM3 or HMFG2.

23. The NK cell or NK cell line of claim 18, wherein the tumour-associated MUC-1 glycoform comprises a preponderance of shorter glycans relative to wildtype glycoforms, and wherein the shorter glycans are selected from the group consisting of Tn, sialyl Tn (STn), T, sialyl T (ST) glycans, and combinations thereof.

24. The NK cell or cell line of claim 18, wherein the NK cell or cell line has reduced propensity to form tumours relative to wildtype NK cells or wildtype NK cell lines.

25. The NK cell or NK cell line of claim 24, wherein the NK cell or cell line is rendered incapable of division.

26. The NK cell or cell line of claim 18, wherein the NK cell or cell line is modified to reduce expression of cancer-related MUC-1 glycoforms.

27. The NK cell or NK cell line of claim 18, wherein the NK cell or cell line is further modified to express a mutant TRAIL ligand with at least 10% higher affinity for one or more TRAIL death receptors relative to wildtype TRAIL.

28. The NK cell or NK cell line of claim 27, wherein the mutant TRAIL ligand comprises a D269H/E195R mutation.

29. The NK cell or NK cell line of claim 18, wherein the NK cell or cell line is further modified to reduce function of one or more checkpoint inhibitory receptors.

30. The NK cell or NK cell line of claim 29, wherein the one or more checkpoint inhibitory receptors are selected from the group consisting of CD96 (TACTILE), CD152 (CTLA4), CD223 (LAG-3), CD279 (PD-1), CD328 (SIGLEC7), SIGLEC9, TIGIT, TIM-3, and combinations thereof.

31. The NK cell or NK cell line of claim 18, wherein the NK cell line is a derivative of a KHYG-1 cell line.

32. A method of treating a cancer in a patient, the method comprising administering to the patient a NK cell or NK cell line modified to express a CAR that binds a tumour-associated MUC-1 glycoform with at least 10% increased affinity relative to wildtype MUC-1 glycoforms.

33. The method of claim 32, wherein the cancer is a solid cancer.

34. The method of claim 33, wherein the solid cancer is selected from the group consisting of breast cancer, ovarian cancer and colorectal cancer.

35. The method of claim 32, wherein the cancer is a blood cancer.

36. The method of claim 35, wherein the blood cancer is selected from the group consisting of acute lymphocytic leukemia (ALL), acute myeloid leukemia (AML), chronic lymphocytic leukemia (CLL), chronic myeloid leukemia (CML), Hodgkin's lymphoma, non-Hodgkin's lymphoma, including T-cell lymphomas and B-cell lymphomas, asymptomatic myeloma, smoldering multiple myeloma (SMM), active myeloma and light chain myeloma.

37. A method of treating multiple myeloma in a human patient, the method comprising administering to the human patient a NK cell or NK cell line modified to express a CAR that binds a tumour-associated MUC-1 glycoform with at least 10% increased affinity relative to wildtype MUC-1 glycoforms.


SEQUENCE LISTING (3 sequences)

SEQ ID NO: 1 - DNA, 2439 bp, Human
atgtggcaactgctgctgcctacagctctgctgcttctggtgtccgccgatatcgtggtcacacaagagagcgccctgaccacctctcctggcgaaacagtgaccctgacctgcagatcttctacaggcgccgtgaccacaagcaactacgccaactgggtgcaagagaagcccgatcacctgttcacaggcctgatcggcggcacaaacaatagagcacctggcgtgccagccagattcagcggatctctgatcggagacaaggccgcactgacaatcacaggcgcccagacagaggacgaggccatctacttttgcgccctgtggtacagcaaccactgggttttcggcggaggcaccaagctgacagtgctgggatctgaaggtggcggaggatctggcggaggtggaagcggaggcggaggttctgaagttcagctgcaacaatctggcggcggactggttcaacctggcggctctatgaagctgagctgtgtggccagcggcttcaccttcagcaactactggatgaactgggtccgacagagccccgagaaaggcctggaatgggttgccgagatcagactgaagtccaacaattacgccacacactacgccgagagcgtgaagggcagattcaccatcagccgggacgacagcaagagcagcgtgtacctccagatgaacaacctgagagccgaggacaccggcatctactactgcaccttcggcaacagcttcgcctattggggccagggaaccaccgtgaccgtgtccagcaccttcacctgttttgtcgtgggcagcgacctgaaggatgcccacctgacatgggaagtcgccggcaaagttcctaccggtggcgtggaagaaggcctgctggaaagacacagcaacggcagccagagccagcacagcagactgacactgcctagaagcctgtggaatgccggcaccagcgtgacctgcacactgaatcatcctagcctgcctccacagagactgatggccctgagagaacctgctgctcaggcccctgtgaagctgtccctgaatctgctcgccagcagcgatcctcctgaagccgccaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtcgaggtgcacaacgccaagacaaagcccagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagagaaccccaggtgtacacactgcctccaagcagagatgagctgaccaagaaccaggtgtccctgacatgcctggtcaagggcttctacccctccgatatcgccgtggaatgggagagcaatggacagcccgagaacaactacaagacaacccctcctgtgctggactccgacggctcattcttcctgtacagcaaactgaccgtggacaagtccagatggcagcagggcaacgtgttctcctgcagcgtgatgcacgaggccctgcacttttgggtgctcgtggttgttggcggagtgctggcctgttacagcctgctggttaccgtggccttcatcatcttttgggtccgaagcaagcggagccggctgctgcacagcgactacatgaacatgacccctagacggcccggacctaccagaaagcactaccagccttacgctcctcctagagacttcgccgcctacagaagcagacgggaccaaagactgcctcctgacgctcacaaacctccaggcggcggaagcttcaggacccctatccaagaagaacaggctgacgcccacagcaccctggccaagatccgcgtgaagttctccagatccgccgacgctcctgcctatcagcagggacagaaccagctgtacaacgagctgaacctggggagaagagaagagtacgacgtgctggataagcggagaggcagagatcctgagatgggcggaaagccccagcggagaaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggctctgcctcca

SEQ ID NO: 2 - protein, 813 aa, Human
MWQLLLPTALLLLVSADIVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVLGSEGGGGSGGGGSGGGGSEVQLQQSGGGLVQPGGSMKLSCVASGFTFSNYWMNWVRQSPEKGLEWVAEIRLKSNNYATHYAESVKGRFTISRDDSKSSVYLQMNNLRAEDTGIYYCTFGNSFAYWGQGTTVTVSSTFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAANVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHFWVLVVVGGVLACYSLLVTVAFIIFWVRSKRSRLLHSDYMNMTPRRPGPTRKHYQPYAPPRDFAAYRSRRDQRLPPDAHKPPGGGSFRTPIQEEQADAHSTLAKIRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPP

SEQ ID NO: 3 - protein, 24 aa, Human
TAPPAHGVTSAPDTRPAPGSTAPP